(19) United States
(12) Patent Application Publication
(10) Pub. No.: US 2005/0203084 A1
(43) Pub. Date: Sep. 15, 2005

(54) Protease inhibitors

(76) Inventors: Jae U. Jeong, Collegeville, PA (US); Dennis S. Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/515,117

(22) PCT Filed: May 21, 2003
(86) PCT No.: PCT/US03/16130

§ 371 Date: Nov. 18, 2004

(30) Foreign Application Priority Data
May 22, 2002 (US) 60382518

(51) Int. Cl. A61K 31/551; C07D 487/02; C07D 487/12

(52) U.S. Cl. 514/220; 540/559

(57) ABSTRACT

This invention relates to certain substituted substituted amides of formula I 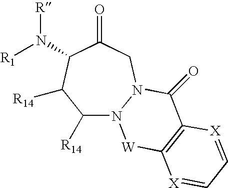 as defined herein which are protease inhibitors.

### BACKGROUND OF THE INVENTION

This invention relates in general to certain substituted 5,8,13-dioxo-5,8,9,10,11,13-hexahydro-7H-[1,2]diazepino[1,2-b]phthalazine amides and related three-ring heteroaromatic compounds which are protease inhibitors. More particularly they are inhibitors of cysteine and serine proteases, particularly compounds which inhibit cysteine proteases. More specifically these compounds inhibit cysteine proteases of the papain superfamily, including, in particular those of the cathepsin family, most particularly cathepsin K. Such compounds are useful for treating diseases in which cysteine proteases are implicated, especially diseases of excessive bone or cartilage loss, e.g., osteoporosis, periodontitis, and arthritis; and certain parasitic diseases, e.g., malaria.

Cathepsins are a family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has also been variously denoted as cathepsin O or cathepsin O2 in the literature: The designation cathepsin K is considered to be the most appropriate one.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I collagen represents the major structural protein of bone comprising approximately 90% of the protein matrix. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodelling at discrete foci throughout life. These foci, or remodelling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

Several published studies have demonstrated that inhibitors of cysteine proteases are effective at inhibiting osteoclast-mediated bone resorption, and indicate an essential role for cysteine proteases in bone resorption. For example, Delaisse, et al., Biochem. J., 1980, 192, 365, disclose a series of protease inhibitors in a mouse bone organ culture systems and suggest that inhibitors of cysteine proteases (e.g., leupeptin, Z-Phe-Ala-CHN2) prevent bone resorption, while serine protease inhibitors were ineffective. Delaisse, et al., Biochem. Biophys. Res. Commun., 1984, 125, 441, disclose that E-64 and leupeptin are also effective at preventing bone resorption in vivo, as measured by acute changes in serum calcium in rats on calcium deficient diets. Lerner, et al., J. Bone Min. Res., 1992, 7, 433, disclose that cystatin, an endogenous cysteine protease inhibitor, inhibits PTH stimulated bone resorption in mouse calvariae. Other studies, such as by Delaisse, et al., Bone, 1987, 8, 305, Hill, et al., J. Cell. Biochem., 1994, 56, 118, and Everts, et al., J. Cell. Physiol., 1992, 150, 221, also report a correlation between inhibition of cysteine protease activity and bone resorption. Tezuka, et al., J. Biol. Chem., 1994, 269, 1106, Inaoka, et al., Biochem. Biophys. Res. Commun., 1995, 206, 89 and Shi, et al., FEBS Lett., 1995, 357, 129 disclose that under normal conditions cathepsin K, a cysteine protease, is abundantly expressed in osteoclasts and may be the major cysteine protease present in these cells.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

We have now discovered a novel class of substituted 5,8,13-dioxo-5,8,9,10,11,13-hexahydro-7H-[1,2]diazepino[1,2-b]phthalazine amides and similar three-ring-based compounds which are protease inhibitors, most particularly of cathepsin K.

### SUMMARY OF INVENTION

The present invention provides substituted 5,8,13-dioxo-5,8,9,10,11,13-hexahydro-7H-[1,2]diazepino[1,2-b]phthalazine amides and related three-ring heteroaromatic compounds for use as protease inhibitors inhibiting the likes of cathepsin K, and which are useful for treating diseases which may be therapeutically modified by altering the activity of such proteases.

Accordingly, in the first aspect, this invention provides a compound according to Formula I. 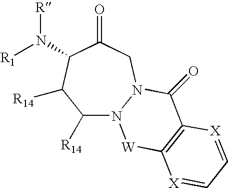
wherein:

R₁ is either formula A or B 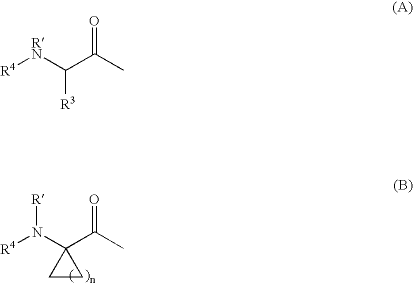
wherein in formula (B), n is an integer from 1 to 5;
R₃ is H, C₁₋₄alkyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;
R₃ and R′ may be connected to form a pyrrolidine, piperidine or morpholine ring;
R₄ is C₁₋₆alkyl, C₃₋₆cycloalkyl-C₀₋₆alkyl, Ar—C₀₋₆alkyl, Het-C₀₋₆alkyl, R₅C(O), R₅—C(S)—, R₅SO₂—, R₅OC(O)—, R₅R₁₂NC(O)—, or R₅R₁₂NC(S)—;
R₅ is H, C₁₋₆alkyl, C₂₋₆alkenyl, C₂₋₆alkynyl, C₃₋₆cycloalkyl-C₀₋₆alkyl, C₂₋₄-alkanonyl, Ar—C₀₋₆aklyl, Het-C₀₋₆alkyl Ar—ArC₀₋₆alkyl, Ar—HetC₀₋₆alkyl, Het-ArC₀₋₆alkyl, or Het-HetC₀₋₆alkyl;
R₁₂ is H, C₁₋₆alkyl, Ar—C₀₋₆alkyl, or Het-C₀₋₆alkyl;
each R₁₄ is independently H, C₁₋₆alkyl, OC₁₋₄alkyl, SC₁₋₄alkyl, N(R₁₂)₂, —CH₂OC₁₋₄alkyl, CH₂SC₁₋₄alkyl, CH₂N(R₁₂)₂, Ar—C₀₋₆alkyl or Het-C₀₋₆alkyl;
R′ is H, C₁₋₆alkyl, Ar—C₀₋₆alkyl, or Het-C₀₋₆alkyl;
R″ is H, C₁₋₆alkyl, Ar—C₀₋₆alkyl, or Het-C₀₋₆alkyl;
W is a bond, CH₂, or C(O);
each X is independently C, or N;
a pharmaceutically acceptable salt, hydrate or solvate thereof.

In another aspect, this invention provides a pharmaceutical composition comprising a compound according to Formula I and a pharmaceutically acceptable carrier, diluent or excipient.

In yet another aspect, this invention provides intermediates useful in the preparation of the compounds of Formula I.

In still another aspect, this invention provides a method of treating diseases in which the disease pathology may be therapeutically modified by inhibiting proteases, particularly cysteine and serine proteases, more particularly cysteine proteases, even more particularly cysteine proteases of the papain superfamily, yet more particularly cysteine proteases of the cathepsin family, most particularly cathepsin K.

In a particular aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis; and for treating certain parasitic diseases, such as malaria.

### DETAILED DESCRIPTION

Definitions and Preferred Embodiments

The present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

“Hydrogen” or “H” includes all of its possible isotopes, including deuterium and tritium.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR₁₅, C(O)R₁₅, SR₁₅, S(O)R₁₅, S(O)₂R₁₅, N(R₁₅)₂ R₁₄NC(O)OR₁₆, CO₂R₁₅, CO₂N(R₁₅)₂′ N(C═NH)NH₂, Het, C₃₋₆-cycloalkyl, and Ar; where R₁₆ is selected from the group consisting of: H, C₁₋₆alkyl, C₂₋₆-alkenyl, C₂₋₆alkynyl, C₃₋₆cycloalkyl-C₀₋₆alkyl, Ar—C₀₋₆alkyl and Het-C₀₋₆alkyl; and R₁₅ is selected from the group consisting of: H, C₁₋₆alkyl, Ar—C₀₋₆alkyl, and Het-C₀₋₆alkyl.

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkanonyl” as applied herein is meant to include unsubstituted and substituted acetyl, propanonyl, butanonyl, pentanonyl, and hexanonyl

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆alkylAr, C₁₋₆alkyl, OR₁₇, N(R₁₇)₂, SR₁₇, S(O)R₁₇, S(O)₂R₁₇, CF₃, NO₂, CN, CO₂R₁₇, CON(R₁₇), F, Cl, Br and I, where R₁₇ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8-naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

“Ar” or “aryl” means phenyl or naphthyl, optionally substituted by one or more of Ph-C₀₋₆alkyl; HetC₀₋₆alkyl; C₁₋₆alkoxy; Ph-C₀₋₆alkoxy; Het-C₀₋₆alkoxy; OH, (CH₂)₁₋₆NR₁₅R₁₆; O(CH₂)₁₋₆NR₁₅R₁₆; C₁₋₆alkyl, OR₁₇, N(R₁₇)₂, SR₁₇, CF₃, NO₂, CN, CO₂R₁₇, CON(R₁₇), F, Cl, Br or I; where R₁₅ and R₁₆ are H, C₁₋₆alkyl, Ph-C₀₋₆alkyl, naphthyl-C₀₋₆alkyl or Het-C₀₋₆-alkyl; and R₁₇ is phenyl, naphthyl, or C₁₋₆alkyl.

“Ar—Ar” means aryl covalently linked to a second aryl. Examples of “Ar—Ar” include biphenyl or naphythyl-pheny or phenyl-naphthyl.

“Ar—Het” means an aryl group covalently linked to a heterocycle. Examples of “Ar-Het” include phenyl-piperidine, phenyl-piperazine, phenyl-2-oxopiperazine, naphthyl-piperidine, naphthyl-piperazine, and naphhyl-2-oxopiperazine.

“Het-Ar” means a heterocycle covalently linked to a aryl group. Examples of such “Het-Ar” include piperidinyl-phenyl, piperazinyl-phenyl, 2-oxopiperazinyl-phenyl, piperidinyl-naphthyl, piperazinyl-naphthyl, and 2-oxpiperazinyl-naphthyl.

“Het-Het” means a heterocycle covalently linked to a second heterocycle. Examples of such “Het-Het” include bipyridine, pyridinyl-piperidine, pyridinyl-piperazine, pyridinyl-2-oxopiperazine, thiophenyl-piperidine, thiophenyl-piperazine, and thiophnyl-2-oxopiperazine.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′-(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

Preferred Embodiments

In compounds of Formula I, when R₁ is 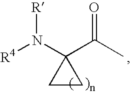
n is preferably 4, to provide 1-amino-1-acyl cyclohexane compounds. The cycloalkyl ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

The cycloalkyl ring is more preferably unsubstituted.

In compounds of Formula I, when R₁ is 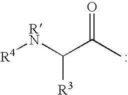
R₃ is H, C₁₋₆alkyl, C₃₋₆cycloalkyl-C₀₋₆alkyl, C₂₋₆alkenyl, C₂₋₆alkynyl, Het-C₀₋₆alkyl, Ar—C₀₋₆alkyl, Ar—ArC₀₋₆alkyl, Ar—HetC₀₋₆alkyl, Het-ArC₀₋₆alkyl, or Het-HetC₀₋₆alkyl.
R₃ is preferably H, C₃₋₆cycloalkyl-C₀₋₆alkyl, Ar—C₀₋₆alkyl, or C₁₋₆alkyl.
R₃ is more preferably H, methyl, ethyl, n-propyl, prop-2-yl, n-butyl, isobutyl, but-2-yl, cyclopentyl, cyclopropylmethyl, cyclohexylmethyl, 2-methanesulfinyl-ethyl, 1-hydroxyethyl, toluyl, naphthalen-2-ylmethyl, benzyloxymethyl, or hydroxymethyl.
R₃ is even more preferably toluyl, isobutyl or cyclohexylmethyl.
R₃ is most preferably isobutyl.
R₄ is H, C₁₋₆alkyl, C₃₋₆cycloalkyl-C₀₋₆alkyl, Ar—C₀₋₆alkyl, Het-C₀₋₆alkyl, R₅C(O)—, R₅C(S)—, R₅SO₂—, R₅OC(O)—, R₅R₁₂NC(O)—, or R₅R₁₂NC(S)—.
R₄ is more preferably R₅OC(O)—, R₅C(O)— or R₅SO₂—.
R₄ is most preferably R₅C(O)—.

In some embodiments, R₄ is preferably methanesulfonyl.

Preferably R₅ is C₁₋₆alkyl, C₂₋₆alkenyl, C₃₋₆cycloalkyl-C₀₋₆alkyl, C₂₋₆alkanonyl, Ar—C₀₋₆alkyl or Het-C₀₋₆alkyl.

More preferably, and especially when R₄ is R₅C(O)—, where R₅ is methyl, especially halogenated methyl, more especially trifluoromethyl, especially C₁₋₆alkoxy and aryloxy substituted methyl, more especially phenoxy-methyl, 4-fluoro-phenoxy-methyl, especially heterocycle substituted methyl, more especially 2-thiophenyl-methyl;
butyl, especially aryl substituted butyl, more especially 4-(4-methoxy)phenyl-butyl;
isopentyl;
cyclohexyl;
pentanonyl, especially 4-pentanonyl;
butenyl, especially aryl substituted butenyl, more especially 4,4-bis(4-methoxyphenyl)but-3-enyl;
phenyl, especially phenyl substituted with one or more halogens, more especially 3,4-dichlorophenyl and 4-fluorophenyl, especially phenyl substituted with one or more C₁₋₆ alkoxy or aryloxy groups, more especially 3,4-dimethoxy-phenyl, 3-benzyloxy-4-methoxy-phenyl, especially phenyl substituted with one or more sulfonyl groups, more especially 4-methanesulfonyl-phenyl;
benzyl;
naphthalenyl, especially naphthylen-2-yl;
benzo[1,3]dioxolyl, especially benzo[1,3]dioxol-5-yl, furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl;
tetrahydrofuranyl, especially tetrahydrofuran-2-yl;
benzofuranyl, especially benzofuran-2-yl, and especially C₁₋₆alkoxy substituted benzofuranyl, more especially 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; 7-methoxybenzofuran-2-yl, 5-methoxy-benzofura-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl;
benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;
quinolinyl, especially quinolin-2-yl, quinolin-3-yl, quinolin-4-yl, quinolin-6-yl, or quinolin-8-yl;
quinoxalinyl, especially quinoxalin-2-yl;
1,8-naphthyridinyl, especially 1,8-naphthyridin-2-yl;
indolyl, especially indol-2-yl, especially indol-6-yl, indol-5-yl, especially C₁₋₆alkyl substituted indolyl, more especially N-methyl-indol-2-yl;
pyridinyl, especially pyridin-2-yl, pyridin-5-yl, especially 1-oxy-pyridin-2-yl, especially C₁₋₆alkyl substituted pyridinyl, more especially 2-methyl-pyridin-5-yl;
furo[3,2-b]pyridinyl, especially furo[3,2-b]pyridin-2-yl, and C₁₋₆alkyl substituted furo[3,2-b]pyridinyl, especially 3-methyl-furo[3,2-b]pyridin-2-yl;
thiophenyl, especially thiophen-3-yl, especially C₁₋₆alkyl substituted thiophenyl, more especially 5-methyl-thiophen-2-yl, especially halogen substituted thiophenyl, more especially 4,5-dibromo-thiophen-2-yl;
thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl;
isoxazolyl, especially isoxazol-4-yl, especially C₁₋₄alkyl substituted isoxazolyl, more especially 3,5-dimethyl-isoxazol-4-yl;
oxazolyl, especially oxazol-4-yl, more especially 5-methyl-2-phenyl oxazol-4-yl, or 2-phenyl-5-trifluoromethyl-oxazol-4-yl.

When R₄ is R₅SO₂, R₅ is preferably pyridin-2-yl or I-oxo-pyridin-2-yl.
R′ is preferably H or naphthalen-2-yl-methyl. Most preferably R′ is H.
R₁₄ is preferably H, C₁₋₆alkyl, especially is methyl, ethyl, propyl, butyl, pentyl or hexyl, more especially methyl. R₁₄ is preferably H.

Compounds of Formula I where R″ is H are preferred.

More preferred are compounds of Formula I wherein:
R₁ is 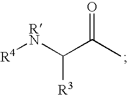
   where:
   R₃ is H, C₁₋₆alkyl, C₃₋₆cycloalkyl-C₀₋₆alkyl, or Ar—C₀₋₆alkyl;
   R₄ is R₅C(O)—, R₁₄SO₂—, or R₅OC(O)—;
   R₅ is C₁₋₆alkyl, C₂₋₆alkenyl, C₃₋₆cycloalkyl-C₀₋₆alkyl, C₂₋₆alkanonyl, Ar—C₀₋₆alkyl or Het-C₀₋₆alkyl;
   R₁₂ is H, C₁₋₆alkyl, Ar—C₀₋₆alkyl, or Het-C₀₋₆alkyl;
   each R₁₄ is independently H, C₁₋₆alkyl, C₂₋₆alkenyl, C₃₋₆cycloalkyl-C₀₋₆alkyl, C₂₋₆alkanonyl, Ar—C₀₋₆alkyl or Het-C₀₋₆alkyl;
   R′ is H; and
   R″ is H.

Particularly preferred are such compounds wherein R₃ is isobutyl.

Still more preferred are compounds of Formula I wherein:
R₁ is 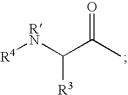
   R₃ is H, C₁₋₆alkyl, C₃₋₆cycloalkyl-C₀₋₆alkyl, or Ar—C₀₋₆alkyl;
   R₄ is R₅OC(O)—, R₅C(O)— and R₅SO₂—;
   R₅ is C₁₋₆alkyl, C₂₋₆alkenyl, C₃₋₆cycloalkyl-C₀₋₆alkyl, C₂₋₆alkanonyl, Ar—C₀₋₆alkyl or Het-C₀₋₆alkyl;
   R′ is H; and
   R″ is H;

Yet more preferred are compounds of Formula I wherein:
R₁ is 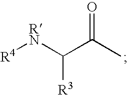
R₃ is H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, or hydroxymethyl;
R₄ is R₅C(O)—;
R₅ is hydrogen, methyl, especially halogenated methyl, more especially trifluoromethyl, especially C₁₋₆alkoxy and aryloxy substituted methyl, more especially phenoxy-methyl, 4-fluoro-phenoxy-methyl, especially heterocycle substituted methyl, more especially 2-thiophenyl-methyl; butyl, especially aryl substituted butyl, more especially 4-(4-methoxy)phenyl-butyl; isopentyl; cyclohexyl; pentanonyl, especially 4-pentanonyl; butenyl, especially aryl substituted butenyl, more especially 4,4-bis(4-methoxyphenyl)-but-3-enyl; phenyl, especially phenyl substituted with one or more halogens, more especially 3,4-dichlorophenyl and 4-fluorophenyl, especially phenyl substituted with one or more C₁₋₆alkoxy or aryloxy groups, more especially 3,4-dimethoxy-phenyl, 3-benzyloxy-4-methoxy-phenyl, especially phenyl substituted with one or more sulfonyl groups, more especially 4-methanesulfonyl-phenyl; benzyl; naphthylen-2-yl; benzo[1,3]dioxolyl, especially benzo[1,3]dioxol-5-yl, furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl; tetrahydrofuran-2-yl; benzofuranyl, especially benzofuran-2-yl, and especially C₁₋₆alkoxy substituted benzofuranyl, more especially 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl, 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl; benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl; quinolinyl, especially quinolin-2-yl, quinolin-3-yl, quinolin-4-yl, quinolin-6-yl, and quinolin-8-yl; quinoxalinyl, especially quinoxalin-2-yl; 1,8-naphthyridinyl, especially 1,8-naphthyridin-2-yl; indolyl, especially indol-2-yl, especially indol-6-yl, indol-5-yl, especially C₁₋₆alkyl substituted indolyl, more especially N-methyl-indol-2-yl; pyridinyl, especially pyridin-2-yl, pyridin-5-yl, especially 1-oxy-pyridin-2-yl, especially C₁₋₆alkyl substituted pyridinyl, more especially 2-methyl-pyridin-5-yl; furo[3,2-b]pyridinyl, especially furo[3,2-b]pyridin-2-yl, and C₁₋₆alkyl substituted furo[3,2-b]pyridinyl, especially 3-methyl-furo[3,2-b]pyridin-2-yl; thiophenyl, especially thiophen-3-yl, especially C₁₋₆alkyl substituted thiophenyl, more especially 5-methyl-thiophen-2-yl, especially halogen substituted thiophenyl, more especially 4,5-dibromo-thiophen-2-yl; thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl; isoxazolyl, especially isoxazol-4-yl, especially C₁₋₆alkyl substituted isoxazolyl, more especially 3,5-dimethyl-isoxazol-4-yl; or oxazolyl, especially oxazol-4-yl, more especially 5-methyl-2-phenyl oxazol-4-yl, 2-phenyl-5-trifluoromethyl-oxazol-4-yl; and
R′ is H.

Even yet more preferred are compounds of Formula I wherein:
R₁ is 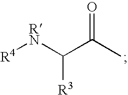
R₃ is C₁₋₆alkyl;
R₄ is R₅C(O);
R₅ is Het-C₀₋₆alkyl;
R′ is H; and
R″ is H

Still yet more preferred are compounds of Formula I wherein:
R₁ is 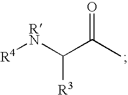
R₃ is isobutyl;
R₄ is R₅C(O);
R₅ is hydrogen, 5-methoxybenzofuran-2-yl, benzo[b]thiophen-2-yl, 3-methyl-benzofuran-2-yl, thieno[3,2-b]thiophen-2-yl, benzofuran-2-yl, furo[3,2-b]pyridin-2-yl, 3-methyl-furo[3,2-b]pyridin-2-yl; preferably benzofuran-2-yl, furo[3,2-b]pyridin-2-yl, or 3-methyl-furo[3,2-b]pyridin-2-yl; most preferably benzofuran-2-yl.
R′is H;
R″ is H;
W is C(O); and
each of X is carbon.
   Synthetic Methods

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺ and NH₄⁺ are specific examples of cations present in pharmaceutically acceptable salts. Halides, sulfates, phosphates, alkanoates (such as acetate and trifluoroacetate), benzoates, and sulfonates (such as mesylate) are examples of anions present in pharmaceutically acceptable salts.

This invention also provides a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, or aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

Utility of the Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Parasites known to utilize cysteine proteases in their life cycle (and the diseases caused by these parasites) include Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lambia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria. The compounds of the present invention are suitable for treating diseases caused by these parasites which may be therapeutically modified by altering the activity of cysteine proteases. In particular, the present compounds are useful for treating malaria by inhibiting falcipain.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

The present method provides treatment of diseases (in parentheses) caused by infection by Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lambia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria by inhibiting cysteine proteases of the papain superfamily by administering to a patient in need thereof, particularly an animal, more particularly a mammal, most particularly a human being; one or more of the above-listed compounds.

Most particularly, the present invention provides a method of treating malaria, caused by infection with Plasmodium falciparum, by the inhibition of falcipain by administering to a patient in need thereof, particularly an animal, more particularly a mammal, most particularly a human being, one or more of the above-listed compounds.

The present method may be practiced by administering the above-listed compounds alone or in combination, with each other, or with other therapeutically effective compounds.

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., alendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

Bioassay

The compounds of this invention may be tested in one of several biological assays to determine the concentration of compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):
v=VₘA/[Kₐ(1+I/K_{i,app})+A] (1)
where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:
[AMC]=vₛₛt+(v₀−vₛₛ) [1−exp(−k_{obs}t)]/k_{obs} (2)
where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or kobs/[I]) describing the time-dependent inhibition. A complete discussion of this kinetic treatment has been fully described (Morrison et al., Adv. Enzymol. Relat. Areas Mol. Biol., 1988, 61, 201).
Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 min on ice The cell suspension was mixed frequently.

The cells were washed ×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 Thin on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated ×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/litre of sodium bicarbonate. 3 mL aliquots of the cell suspension (per treatment) were decanted into 15 mL centrifuge tubes. These cells were pelleted by centrifugation. To each tube 3 mL of the appropriate treatment was added (diluted to 50 uM in the EMEM medium). Also included were appropriate vehicle controls, a positive control (87MEM1 diluted to 100 ug/mL) and an isotype control (IgG2a diluted to 100 ug/mL). The tubes were incubate at 37° C. for 30 min.

Aliquots (0.5 mL) of the cells were seeded onto sterile dentine slices in a 48-well plate and incubated at 37° C. for 2 h. Each treatment was screened in quadruplicate. The slices were washed in six changes of warm PBS (10 mL/well in a 6-well plate) and then placed into fresh treatment or control and incubated at 37° C. for 48 h. The slices were then washed in phosphate buffered saline and fixed in 2% glutaraldehyde (in 0.2M sodium cacodylate) for 5 min., following which they were washed in water and incubated in buffer for 5 min at 37° C. The slices were then washed in cold water and incubated in cold acetate buffer/fast red garnet for 5 min at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FFIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

Methods of Preparation and Specific Examples

Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

The following Scheme I illustrates one process for preparing the compounds of this invention.

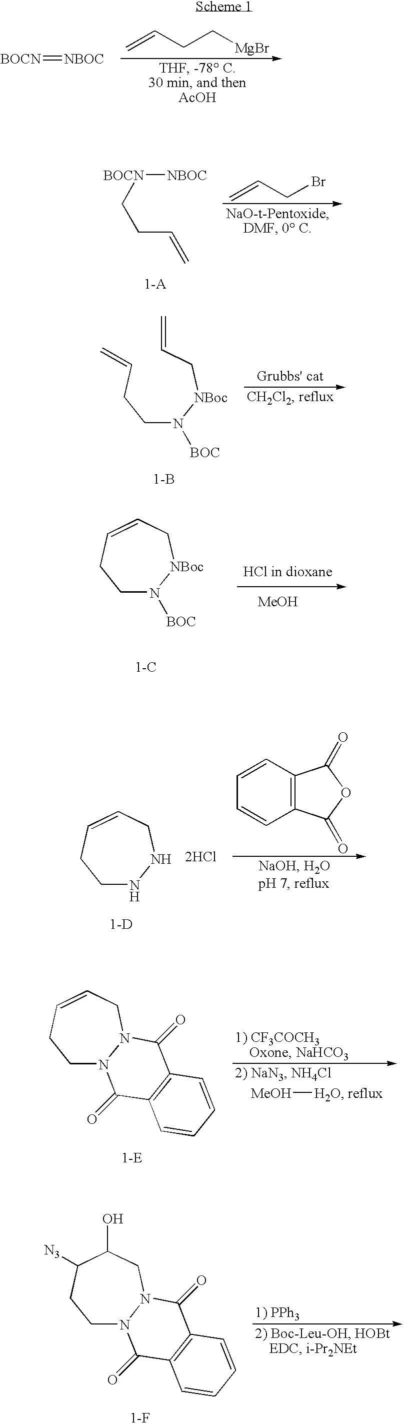

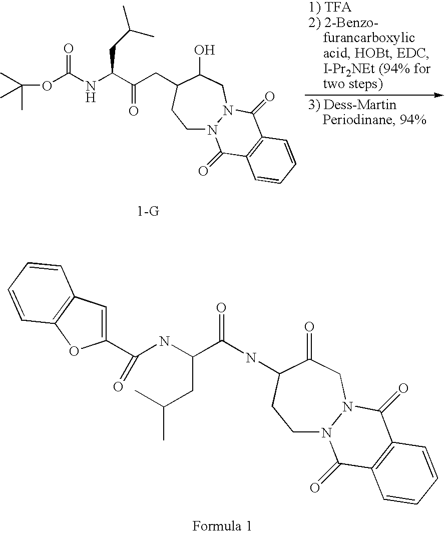

This scheme is presented for purposes of illustrating a method for making the compounds of this invention. In a general sense, the three-ring structure, illustrated by the 8,11-dihydro-7H-[1,2]diazepino[1,2-b]phthalazine-5,13-dione, is prepared first, then amidated by published procedures. As illustrated here, starting material di-tert-butyl azodicarboxylate (BOCN═NOBC) is treated with an allyl Grignard or a similar allyl organometallic reagent at reduced temperature to form hydrazine-containing butyl ester 1-A. The hydrazine is then alkylated with an allyl halide. Grubbs' catalyst is then used to form the diazepine ring (compound 1-C) by ring-closing metathesis. The nitrogen protecting groups are then hydrolyzed with acid (1-D) and the resulting diazepine is treated with an anhydride to form the tricycylic compound (1-E). The azapine-ring double bond is epoxidated and converted into the amino alcohol by opening with sodium azide followed by reduction with, for example, triphenylphosphine. Acylation of the free amine with Boc-leucine and a coupling reagent followed by deprotection of the Boc group with acid, and acylation with a variety of carboxylic acids and coupling reagents such as HBTU or EDC gave intermediate alcohols (e.g. 1-G). Final oxidation with Dess-Martin periodinane gave the final product.

This set of steps can be used to make other compounds of formula 1, by simply varing the starting material or the penultimate ester-forming step. In addition the synthetic processes described in the PCT application having publication number WO 01-70232 published 27 Sep. 2001 can be used to make compounds of this invention as well. Those chemistries are incorporated herein by reference in full.

The following specific examples are provided to illustrate the invention further. They are representative examples, and are not intended to limit the invention. Reference is made to the claims for what is reserved to the inventors hereunder.

### EXAMPLES

### Example 1

### Preparation of benzofuran-2-carboxylic acid [(S)-methyl-1-((S)-5,8,13-trioxo-5,8,9,10,11,13-hexahydro-7H-[1,2]diazepino[1,2-b]phthalazin-9-ylcarbamoyl)-butyl]-amide

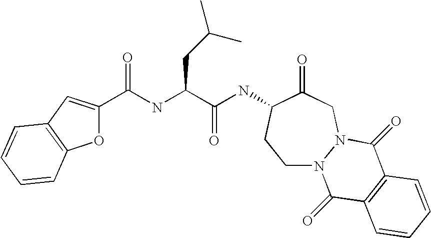

### 1a. N-But-3-enyl-hydrazine-1,2-dicarboxylic acid di-tert-butyl ester

To a yellowish solution of di-tert-butyl azodicarboxylate (5.3 g, 23.02 mmol) in THF (60 ml) at −78° C. was dropwisely added 3-butenylmagnesium bromide (0.5 M in THF, 55.2 ml, 27.62 mmol) during 20 min. The yellow color was disappeared during addition. After another 30 min at −78° C., the reaction was quenched with AcOH (3.4 ml, 59.84 mmol). After THF was evaporated under the reduced pressure, water (150 ml) was added to the residue and extracted with EtOAc (100 ml) followed by washing with 2N HCl, sat'd NaHCO₃, and brine. The organic solution was dried over MgSO₄, filtered, and evaporated and then the residue was purified by flash column chromatography on silica gel (15% EtOAc/hexane) to provide 5.7 g (87%) of the title compound; ¹H NMR (CDCl₃): δ 1.46 and 1.50 (two s, 18H), 2.30-2.42 (m, 2H), 3.47-3.63 (m, 2H), 5.02-5.14 (m, 2H), 5.75-5.87 (m, 1H), 6.22-6.38 (brs, 1H).

### 1b. N′-Allyl-N-but-3-enyl-hydrazine-1,2-dicarboxylic acid di-tert-butyl ester

To a solution of N-but-3-enyl-hydrazine-1,2-dicarboxylic acid di-tert-butyl ester (286 mg, 1 mmol) in DMF (3 ml) at 0° C. were added sodium tert-pentoxide (165 mg, 1.5 mmol) and allyl bromide (0.1 ml, 1.2 mmol). After 20 min at 0° C., 20 ml of water was added to the reaction mixture followed by extraction with methyl tert-butyl ether (20 ml×2), washing with brine, drying over MgSO₄, filtration, and evaporation under the reduced pressure. The residue was purified by a plug of silica gel (5% EtOAc/hexane) to give 310 mg (94%) of the product; ¹H NMR (CDCl₃): δ 1.43-1.54 (m, 9H), 2.29-2.41 (m, 2H), 3.23-4.62 (m, 2H), 3.82-4.29 (m, 2H), 4.97-5.22 (m, 4H), 5.72-5.97 (m, 2H).

### 1c. 4,7-Dihydro-3H-[1,2]diazepine-1,2-dicarboxylic acid di-tert-butyl ester

Nζ-Allyl-N-but-3-enyl-hydrazine-1,2-dicarboxylic acid di-tert-butyl ester (306 mg, 0.94 mmol) was dissolved in CH₂Cl₂ (5 ml) and a stream of argon gas was bubbled into the reaction mixture for 5 minutes. Then bis(tricyclohexylphosphine)benzylidine ruthenium (IV) dichloride (Strem Chemicals, Grubbs' catalyst, 6 mg, 0.0066 mmol) was added and the reaction mixture was refluxed for 1.5 hr. The reaction mixture was cooled to rt under argon, then was concentrated in vacuo by rotary evaporation, then was chromatographed on silica gel (3% EtOAc/hexane) to give the desired product (261 mg, 93%); ¹H NMR (CDCl₃): δ 1.32-1.46 (m, 18H), 1.96-2.09 (m, 1H), 2.30-2.44 (m, 1H), 3.28-3.80 (m, 3H), 4.39-4.67 (m, 1H), 5.31-5.40 (m, 1H), 5.53-5.67 (m, 1H).

### 1d. 8,11-Dihydro-7H-[1,2]diazepino[1,2-b]phthalazine-5,13-dione

4M HCl in dioxane (3 ml, 12 mmol) was added to a solution of 4,7-dihydro-3H-[1,2]diazepine-1,2-dicarboxylic acid di-tert-butyl ester (268 mg, 0.9 mmol) and the reaction mixture was stirred for 2 hr at rt followed by evaporation under the reduced pressure. The residue was dried in vacuo and suspended in toluene (3 ml). Phthalic anhydride (150 mg, 1.0 mmol) and NaHCO₃ (166 mg, 2.0 mmol) were added and the reaction mixture was heated to reflux with Dean-Stark trap for 18 hr. After cooling down to RT, silica gel was added to the reaction mixture. The silica gel slurry was dried under the reduced pressure and subjected to flash column chromatography on silica gel to give 163 mg (80%) of the title compound; ¹H NMR (CDCl₃): δ 2.56-2.71 (m, 2H), 4.64-4.78 (m, 2H), 4.95-5.09 (m, 2H), 5.84-5.97 (m, 2H), 7.74-8.87 (m, 2H), 8.30-8.41 (m, 2H); LCMS (MH⁺); 229.2.

### 1e. 9-Azido-8-hydroxy-8,9,10,11-tetrahydro-7H-[1,2]diazepino[1,2-b]phthalazine-5,13-dione

To a solution 8,11-dihydro-7H-[1,2]diazepino[1,2-b]phthalazine-5,13-dione (160 mg, 0.7 mmol) in acetonitrile (6 ml)-water (3 ml) were added CF₃COCH₃ (0.7 ml, 5.61 mmol) and NaHCO₃ (176 mg, 2.1 mmol) at 0° C. Oxone (860 mg, 1.4 mmol) was slowly added to the reaction mixture during 20 min. After stirring for 2 hr at 0° C., additional oxone (860 mg) and NaHCO₃ (176 mg) were added. After stirring for another 2 hr at 0° C., the volume of reaction mixture was reduced to about 1/3 under the reduced pressure. H₂O (30 ml) was added to the residue and extracted with CHCl₃ (30 ml×2). The combined organic layer was washed with 20% aq. Na₂S₂O₃ (30 ml), sat'd NaHCO₃ (30 ml), and brine (20 ml) and dried over MgSO₄. After evaporation and drying under the reduced pressure, the crude epoxides were dissolve in MeOH (5 ml) and water (0.5 ml).

Sodium azide (137 mg, 2.1 mmol) and ammonium chloride (53.5 mg, 2.1 mmol). The reaction mixture was refluxed for 2 hr. After cooling down to RT, silica gel was added to the reaction mixture. The silica gel slurry was dried under the reduced pressure and subjected to flash column chromatography on silica gel to give 92 mg (46% for two steps) of the title compound and 11 mg (6%) of regio-isomer; LCMS (MH⁺): 288.2.

### 1f. [(S)-1-(8-Hydroxy-5,13-dioxo-5,8,9,10,11,13-hexahydro-7H-[1,2]diazepino[1,2-b]phthalazin-9-ylcarbamoyl)-3-methyl-butyl]-carbamic acid tert-butyl ester

Triphenylphosphine (126 mg, 0.48 mmol) was added to a solution of 9-azido-8-hydroxy-8,9,10,11-tetrahydro-7H-[1,2]diazepino[1,2-b]phthalazine-5,13-dione (92 mg, 0.32 mmol) in THF (5 ml) and H₂O (0.02 ml), then was heated to 45° C. for overnight. The reaction mixture was evaporated and then diluted with toluene (10 ml×2) and was azeotroped int vacuo by rotary evaporation twice. After drying under the vacuum, the residue was dissolved in DMP (3 ml) followed by the addition of Boc-Leucine-hydrate (96 mg, 0.384 mmol), 1-hydroxybenzotriazole (56 mg, 0.416 mmol), 1-(3-dimethylaminopropyl)-3-ethylcarbodiimide HCl (79 mg, 0.416 mmol), and N,N-diisopropylethylamine (0.11 ml, 0.64 mmol). The reaction mixture was stirred for overnight at rt, then was diluted with EtOAc, washed with cold 1N HCl, sat'd NaHCO₃, and brine, dried over magnesium sulfate, filtered, concentrated in vacuo by rotary evaporation, and chromatographed on silica gel (2% to 3% MeOH/CH₂Cl₂) to yield the title compound (129 mg, 85% for two steps); ¹H NMR (CDCl₃): δ 0.90-1.03 (m, 6H), 1.43 and 1.45 (two s, 9H), 1.51-1.92 (m, 4H), 2.29-2.42 (m, 1H), 3.83-4.20 (m, 5H), 4.84-5.33 (m, 3H), 6.90 (brs, 1H), 7.79-7.82 (m, 2H), 8.27-8.38 (m, 2H); LCMS (MH⁺): 475.2.

### 1g. Benzofuran-2-carboxylic acid [(S)-1-(8-hydroxy-5,13-dioxo-5,8,9,10,11,13-hexahydro-7H-[1,2]diazepino[1,2-b]phthalazin-9-ylcarbamoyl)-3-methyl-butyl]-amide

4M HCl in dioxane (1 ml, 4 mmol) was added to a solution of [(S)-1-(8-hydroxy-5,13-dioxo-5,8,9,10,11,13-hexahydro-7H-[1,2]diazepino[1,2-b]phthalazin-9-ylcarbamoyl)-3-methyl-butyl]-carbamic acid tert-butyl ester (129 mg, 0.272 mmol) in MeOH (1 ml) at RT. The reaction mixture was stirred for 1 hr at RT, then was concentrated under the reduced pressure. After drying under the vacuum, the residue was dissolved in DMF (2 ml) followed by the addition of benzofuran-2-carboxylic acid (54 mg, 0.33 mmol), 1-hydroxybenzotriazole (47 mg, 0.35 mmol), 1-(3-dimethylaminopropyl)-3-ethylcarbodiimide HCl (67 mg, 0.35 mmol), and N,N-diisopropylethylamine (0.12 ml, 0.66 mmol). The reaction mixture was stirred for overnight at rt, then was diluted with EtOAc (50 ml), washed with cold 1N HCl (30 ml), sat'd NaHCO₃ (30 ml), and brine (30 ml), dried over magnesium sulfate, filtered, concentrated in vacuo by rotary evaporation, and chromatographed on silica gel (50% to 90% EtOAc/hexane) to yield the title compound (131 mg, 94% for two steps); LCMS (MH⁺): 519.2.

### 1 h. Benzofuran-2-carboxylic acid [(S)-methyl-1-((S)-5,8,13-trioxo-5,8,9,10,11,13-hexahydro-7H-[1,2]diazepino[1,2-b]phthalazin-9-ylcarbamoyl)-butyl]-amide

Dess-Martin periodinane (161 mg, 0.38 mmol) was added to a solution of benzofuran-2-carboxylic acid [(S)-1-(8-hydroxy-5,13-dioxo-5,8,9,10,11,13-hexahydro-7H-[1,2]diazepino[1,2-b]phthalazin-9-ylcarbamoyl)-3-methyl-butyl]-amide (131 mg, 0.25 mmol) in CH₂Cl₂ (10 ml) at RT. After stirring for 2 hr at RT, the reaction was quenched by 20% aq. Na₂S₂O₃ (10 ml). The water layer was extracted with CH₂Cl₂ (10 ml). The combined organic layer was washed with sat'd NaHCO₃ (20 ml) and brine (20 ml), then dried over MgSO₄. After evaporation under the reduced pressure, the residue was purified by flash column chromatograph on silica gel (2% MeOH/CH₂Cl₂) to give 121 mg (94%) of the title compound; ¹H NMR (CDCl₃): δ 1.01 (s, 6H), 1.21-1.87 (m, 5H), 2.79-3.00 (m, 1H), 3.54-3.68 (m, 1H), 4.26 (d, J=19.4 Hz, 1H), 4.62-4.76 (m, 1H), 5.16-5.24 (m, 1H), 5.35-5.44 (m, 1H), 6.04 (d, J=19.4 Hz, 1H), 6.93-7.02 (in 1H), 7.21-7.56 (m, 4H), 7.68 (d, 1H), 7.79-7.90 (m, 2H), 8.30-8.42 (m, 2H); LCMS (MH⁺): 517.2.

1. A compound of Formula I. 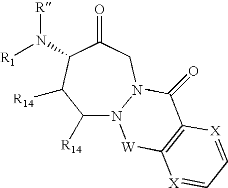
wherein:
R₁ is either formula A or B 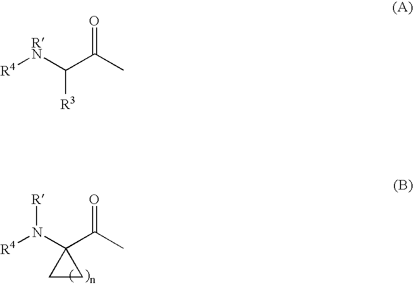
wherein in formula (B), n is an integer from 1 to 5;
R₃ is H, C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆alkyl, Ar—HetC₀₋₆alkyl, Het-ArC₀₋₆alkyl, or Het-HetC₀₋₆alkyl;
R₃ and R′ may be connected to form a pyrrolidine, piperidine or morpholine ring;
R₄ is C₁₋₆alkyl, C₃₋₆cycloalkyl-C₀₋₆alkyl, Ar—C₀₋₆alkyl, Het-C₀₋₆alkyl, R₅C(O), R₅—C(S)—, R₅SO₂—, R₅OC(O)—, R₅R₁₂NC(O)—, or R₅R₁₂NC(S)—;
R₅ is H, C₁₋₆alkyl, C₂₋₆alkenyl, C₂₋₆alkynyl, C₃₋₆cycloalkyl-C₀₋₆alkyl, C₂₋₆-alkanonyl, Ar—C₀₋₆alkyl, Het-C₀₋₆alkyl Ar—ArC₀₋₆alkyl, Ar—HetC₀₋₆alkyl, Het-ArC₀₋₆alkyl, or Het-HetC₀₋₆alkyl;
R₁₂ is H, C₁₋₆alkyl, Ar—C₀₋₆alkyl, or Het-C₀₋₆alkyl;
each R₁₄ is independently H, C₁₋₆alkyl, OC₁₋₄alkyl, SC₁₋₄alkyl, N(R₁₂)₂, —CH₂OC₁₋₄alkyl, CH₂SC₁₋₄alkyl, CH₂N(R₁₂)₂, Ar—C₀₋₆alkyl or Het-C₀₋₆alkyl;
R′ is H, C₁₋₆alkyl, Ar—C₀₋₆alkyl, or Het-C₀₋₆alkyl;
R″ is H, C₁₋₆alkyl, Ar—C₀₋₆alkyl, or Het-C₀₋₆alkyl;
W is a bond, CH₂, or C(O);
each X is independently C, or N;
a pharmaceutically acceptable salt, hydrate or solvate thereof.

2. A compound according to claim 1 wherein R₁ is

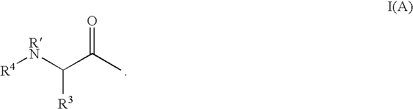

3. A compound according to claim 1 wherein W is C(O) and X is C.

4. A compound according to claim 2 wherein R₃ is C₁₋₆alkyl, C₃₋₆cycloalkyl-C₀₋₆alkyl, or ArC₀₋₆alkyl.

5. A compound according to claim 4 wherein R₃ is H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, and hydroxymethyl.

6. A compound according to claim 4 wherein R₃ is toluyl, isobutyl or cyclohexylmethyl.

7. A compound according to claim 4 wherein R₃ is isobutyl.

8. A compound according to claim 1 wherein R₄ is R₅C(O)—, R₅C(S)—, R₁₄SO₂—.

9. A compound according to claim 8 wherein R₅ is C₁₋₆alkyl, C₂₋₆alkenyl, C₃₋₆cycloalkyl-C₀₋₆alkyl, C₂₋₆-alkanonyl, Ar—C₀₋₆alkyl, or Het-C₀₋₆alkyl.

10. A compound according to claim 9 wherein R₅ is:
methyl, halogenated methyl, C₁₋₆ alkoxy and aryloxy substituted methyl, heterocycle substituted methyl;
butyl, aryl substituted butyl;
isopentyl;
cyclohexyl;
butenyl, aryl substituted butenyl;
pentanonyl;
phenyl, phenyl substituted with one or more halogens, phenyl substituted with one or more C₁₋₆alkoxy groups, phenyl substituted with one or more sulfonyl groups;
benzyl;
naphthylenyl;
benzo[1,3]dioxolyl;
furanyl, halogen substituted furanyl, aryl substituted furanyl;
tetrahydrofuranyl;
benzofuranyl, C₁₋₆alkoxy substituted benzofuranyl, halogen substituted benzofuranyl, C₁₋₆alkyl substituted benzofuranyl;
benzo[b]thiophenyl, C₁₋₆ alkoxy substituted benzo[b]thiophenyl;
quinolinyl;
quinoxalinyl;
1,8-naphthyridinyl;
indolyl, C₁₋₆alkyl substituted indolyl;
pyridinyl, C₁₋₆alkyl substituted pyridinyl, 1-oxy-pyridinyl;
furo[3,2-b]pyridinyl, C₁₋₆alkyl substituted furo[3,2-b]pyridinyl;
thiophenyl, C₁₋₆alkyl substituted thiophenyl, halogen substituted thiophenyl;
thieno[3,2-b]thiophenyl;
isoxazolyl, C₁₋₆alkyl substituted isoxazolyl; or
oxazolyl.

11. A compound according to claim 10 wherein R₅ is:
4-pentanonyl;
naphthylen-2-yl;
benzo[1,3]dioxol-5-yl,
tetrahydrofuran-2-yl
furan-2-yl;
benzofuran-2-yl;
benzo[b]thiophen-2-yl;
quinolin-2-yl, quinolin-3-yl, quinolin-4-yl, quinolin-6-yl, and quinolin-8-yl;
quinoxalin-2-yl;
1,8-naphthyridin-2-yl;
indol-3-yl, indol-5-yl;
pyridin-2-yl, pyridin-5-yl;
furo[3,2-b]pyridin-2-yl;
thiophen-3-yl;
thieno[3,2-b]thiophene-2-yl;
isoxazol-4-yl; or
oxazol-4-yl.

12. A compound according to claim 2 which is:
benzofuran-2-carboxylic acid [(S)-methyl-1-((S)-5,8,13-trioxo-5,8,9,10,11,13-hexahydro-7H-[1,2]diazepino[1,2-b]phthalazin-9-ylcarbamoyl)-butyl]-amide; or
a pharmaceutically acceptable salt thereof.

13. A pharmaceutical preparation comprising a compound according to claim 1 and a pharmaceutically acceptable excipient.

14. A method for inhibiting a protease comprising administering to a patient in need thereof an effective amount of a compound according to claim 1.

15. A method according to claim 14 wherein said protease is selected from the group consisting of a cysteine protease and a serine protease.

16. A method according to claim 14 wherein said protease is a cysteine protease.

17. A method according to claim 16 wherein said cysteine protease is cathepsin K.

18. A method according to claim 16 wherein the cysteine protease is falcipain.

19. A method of treating a disease characterized by bone loss comprising inhibiting said bone loss by administering to a patient in need thereof an effective amount of a compound according to claim 1.

20. A method according to claim 19 wherein said disease is osteoporosis.

21. A method according to claim 19 wherein said disease is periodontitis.

22. A method according to claim 19 wherein said disease is gingivitis.

23. A method of treating a disease characterized by excessive cartilage or matrix degradation comprising inhibiting said excessive cartilage or matrix degradation by administering to a patient in need thereof an effective amount of a compound according to claim 1.

24. A method according to claim 23 wherein said disease is osteoarthritis.

25. A method according to claim 23 wherein said disease is rheumatoid arthritis.

26. A method of treating a disease characterized by infection by a parasite selected from the group consisting of: Plasmodium falciparum, Trypanosoma cruzi, Trypanosoma Brucei, Leishmania mexicana, Leishmania pifanoi, Leishmania major, Schistosoma mansoni, Onchocerca volvulus, Brugia pahangi, Entamoeba histolytica, Giardia lamblia, the helminths Haemonchus contortus and Fasciola hepatica, the helminths of the genera Spirometra, Trichinella, Necator and Ascaris, and protozoa of the genera Cryptosporidium, Eimeria, Toxoplasma and Naegleria, comprising inhibiting expression of a cysteine protease causing said disease by administering to a patient in need thereof an effective amount of a compound according to claim 1.

27. A method according to claim 26 wherein said disease is selected from the group consisting of: malaria, trypanosomiasis (African sleeping sickness, Chagas disease), leishmaniasis, schistosomiasis, onchocerciasis (river blindness) and giardiasis.

28. A process for the synthesis of a compound according to claim 1 comprising the step of oxidizing a compound of formula II 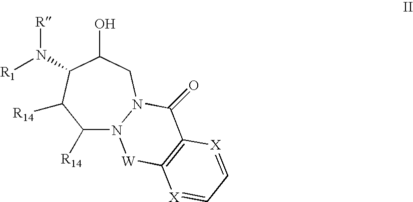
where the R groups are the same as defined in claim 1, with an oxidizing agent to provide compounds of formula I as defined in claim 1 as a mixture of diastereomers.

29. The process of claim 28 wherein the oxidizing agent is sulfur dioxide-pyridine complex or Dess-Martin periodinane.

30. The process of claim 28 further comprising the steps of separating the diasteromers by separating means.

31. The process of claim 30 wherein said separating means is high presssure liquid chromatography (HPLC).
